SEQUENCE LISTING (49 sequences)

SEQ ID NO: 1 - DNA, 53 bp, Artificial Sequence
  Description of Artificial SequencePRIMER
cacctgggccatggccggctgggcccctaagcctaaggcagcttgacttgcag

SEQ ID NO: 2 - DNA, 26 bp, Artificial Sequence
  Description of Artificial SequencePRIMER
gggcttgattggagctcgctcattcc

SEQ ID NO: 3 - DNA, 32 bp, Artificial Sequence
  Description of Artificial SequencePRIMER
gcaggatccgatgcacacaagagtgaggttgc

SEQ ID NO: 4 - DNA, 32 bp, Artificial Sequence
  Description of Artificial SequencePRIMER
tgaggagacggtgaccgtggtcccttggcccc

SEQ ID NO: 5 - DNA, 21 bp, Artificial Sequence
  Description of Artificial SequencePRIMER
gcaaatggcattctgacatcc

SEQ ID NO: 6 - DNA, 21 bp, Artificial Sequence
  Description of Artificial SequencePRIMER
tcggcttattccaggggtgtg

SEQ ID NO: 7 - DNA, 21 bp, Artificial Sequence
  Description of Artificial SequencePRIMER
tcccccgattggtgagaccag

SEQ ID NO: 8 - DNA, 25 bp, Artificial Sequence
  Description of Artificial SequencePRIMER
aaatgatgagatgcctgctgacttg

SEQ ID NO: 9 - DNA, 23 bp, Artificial Sequence
  Description of Artificial SequencePRIMER
atgcgctgttagttcgttacacc

SEQ ID NO: 10 - DNA, 21 bp, Artificial Sequence
  Description of Artificial SequencePRIMER
gaaacatacgttcccaaagag

SEQ ID NO: 11 - DNA, 22 bp, Artificial Sequence
  Description of Artificial SequencePRIMER
cacacccctggaataagccgag

SEQ ID NO: 12 - DNA, 21 bp, Artificial Sequence
  Description of Artificial SequencePRIMER
ggggaggtttgggttgtcatc

SEQ ID NO: 13 - DNA, 20 bp, Artificial Sequence
  Description of Artificial SequencePRIMER
tggcagcattccgtgtggac

SEQ ID NO: 14 - DNA, 24 bp, Artificial Sequence
  Description of Artificial SequencePRIMER
acatttgctgcccacttttcctag

SEQ ID NO: 15 - DNA, 24 bp, Artificial Sequence
  Description of Artificial SequencePRIMER
aaaagcagcgaaatcatccataac

SEQ ID NO: 16 - DNA, 22 bp, Artificial Sequence
  Description of Artificial SequencePRIMER
aggtsmarctgcagsagtcwgg

SEQ ID NO: 17 - DNA, 53 bp, Artificial Sequence
  Description of Artificial SequencePRIMER
aacagttaagcttccgcttgcggccgcggagctggggtcttcgctgtggtgcg

SEQ ID NO: 18 - DNA, 53 bp, Artificial Sequence
  Description of Artificial SequencePRIMER
aacagttaagcttccgcttgcggccgctggttgtggttttggtgtcttgggtt

SEQ ID NO: 19 - DNA, 28 bp, Artificial Sequence
  Description of Artificial SequencePRIMER
gggaattccaataggtggttagcaatcg

SEQ ID NO: 20 - DNA, 26 bp, Artificial Sequence
  Description of Artificial SequencePRIMER
gaccaacgtggtcgcctggcaaaacg

SEQ ID NO: 21 - DNA, 26 bp, Artificial Sequence
  Description of Artificial SequencePRIMER
cgttttgccaggcgaccacgttggtc

SEQ ID NO: 22 - DNA, 30 bp, Artificial Sequence
  Description of Artificial SequencePRIMER
ccccaagcttacatggtcttaagttggcgt

SEQ ID NO: 23 - DNA, 155 bp, Artificial Sequence
  Description of Artificial SequencePLASMID CONSTRUCT
gagctcatcacacaaacaaacaaaacaaaatgatgcttttgcaagccttccttttccttttggctggttttgcagccaaaatatctgcgcaggtgcagctgcaggagtcataatgagggacccaggtcaccgtctcctcataatgacttaagctt

SEQ ID NO: 24 - protein, 36 aa, Artificial Sequence
  Description of Artificial SequencePROTEIN FROM PLASMID CONSTRUCT
AHHTNKQNKMMLLQAFLFLLAGFAAKISAQVQLQES

SEQ ID NO: 25 - DNA, 188 bp, Artificial Sequence
  Description of Artificial SequencePLASMID CONSTRUCT
gagctcatcacacaaacaaacaaaacaaaatgatgcttttgcaagccttccttttccttttggctggttttgcagccaaaatatctgcgcaggtgcagctgcaggagtcataatgagggacccaggtcaccgtctcctcagaacaaaaactcatctcagaagaggatctgaattaatgacttaagctt

SEQ ID NO: 26 - protein, 36 aa, Artificial Sequence; identical to SEQ ID NO: 24
  Description of Artificial SequencePROTEIN FROM PLASMID CONSTRUCT

SEQ ID NO: 27 - DNA, 43 bp, Artificial Sequence
  Description of Artificial SequencePRIMER
gaattaagcggccgcccaggtgaaactgctcgagtcwggggga

SEQ ID NO: 28 - DNA, 42 bp, Artificial Sequence
  Description of Artificial SequencePRIMER
ccctgggtccagtggcagaggagtggcagaggagtcttgttt

SEQ ID NO: 29 - DNA, 24 bp, Artificial Sequence
  Description of Artificial SequencePLASMID CONSTRUCT
caggtccagctgcaggagtctggg

SEQ ID NO: 30 - DNA, 24 bp, Artificial Sequence
  Description of Artificial SequencePLASMID CONSTRUCT
caggtgaaactgctcgagtcwggg

SEQ ID NO: 31 - DNA, 55 bp, Artificial Sequence
  Description of Artificial SequenceSYNTHETIC LINKER
ggtcaccgtctcctcacaggtgcagctgcaggagtcactgtaatgacttaagctt

SEQ ID NO: 32 - DNA, 47 bp, Artificial Sequence
  Description of Artificial SequenceSYNTHETIC INSERT
aagctgctagccaggtgaaactgctcgagcccgggaagcttgaattc

SEQ ID NO: 33 - DNA, 41 bp, Artificial Sequence
  Description of Artificial SequencePRIMER
agctgctagccaggtgaaactgctcgagcccgggaagcttg

SEQ ID NO: 34 - DNA, 41 bp, Artificial Sequence
  Description of Artificial SequencePRIMER
aattcaagcttcccgggctcgagcagtttcacctggctagc

SEQ ID NO: 35 - DNA, 30 bp, Artificial Sequence
  Description of Artificial SequenceSYNTHETIC INSERT
ctcgagaaaagagctagccccggggaattc

SEQ ID NO: 36 - DNA, 24 bp, Artificial Sequence
  Description of Artificial SequencePRIMER
tcgagaaaagagctagccccgggg

SEQ ID NO: 37 - DNA, 24 bp, Artificial Sequence
  Description of Artificial SequencePRIMER
aattccccggggctagctcttttc

SEQ ID NO: 38 - DNA, 47 bp, Artificial Sequence
  Description of Artificial SequenceSYNTHETIC INSERT
ctctcgagaaaagacatcaccatcaccatcacggctcttacgtacag

SEQ ID NO: 39 - DNA, 38 bp, Artificial Sequence
  Description of Artificial SequencePRIMER
tcgagaaaagacatcaccatcaccatcacggctcttac

SEQ ID NO: 40 - DNA, 34 bp, Artificial Sequence
  Description of Artificial SequencePRIMER
gtaagagccgtgatggtgatggtgatgtcttttc

SEQ ID NO: 41 - DNA, 21 bp, Artificial Sequence
  Description of Artificial SequencePRIMER
gactggttccaattgacaagc

SEQ ID NO: 42 - DNA, 60 bp, Artificial Sequence
  Description of Artificial SequencePRIMER
atcgaattctctagatccaccgcctccagaaccgccagtgatcccggcggcggtcacgaa

SEQ ID NO: 43 - protein, 500 aa, Artificial Sequence
  Description of Artificial SequenceLLAMA ANTIBODY FRAGMENT-GFP FUSION PROTEIN
HHHHHHGSYGIHRPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITGGSGGGGSSQVKLLESGGELVQPGGSLKLSCAASGLTFTNYSMGWFRPGPGVDREAVAAISWSGDNTYYVSSVKGRFTISRDNAKNTVYLQMNSLKPQDTAVYYCAVKPDDGWWDYWGQGTQVTVSSQVQLQESGGGLVQAGESLKLSCAASGNTFSGGFMGWYRQAPGKQRELVATINSRGITNYADFVKGRFTISRDNAKKTVYLEMNSLEPEDTAVYYCYTHYFRSYWGQGTQVTVSSEQKLISEEDLN

SEQ ID NO: 44 - protein, 8 aa, Artificial Sequence
  Description of Artificial SequencePROTEIN FROM PLASMID CONSTRUCT
GTQVTVSS

SEQ ID NO: 45 - protein, 19 aa, Artificial Sequence
  Description of Artificial SequencePROTEIN FROM PLASMID CONSTRUCT
GTQVTVSSEQKLISEEDLN

SEQ ID NO: 46 - protein, 13 aa, Artificial Sequence
  Description of Artificial SequencePROTEIN FROM VECTOR LINKER
VTVSSQVQLQESL

SEQ ID NO: 47 - protein, 8 aa, Artificial Sequence
  Description of Artificial SequencePROTEIN FROM PLASMID INSERT
ASQVKLLE

SEQ ID NO: 48 - protein, 6 aa, Artificial Sequence
  Description of Artificial SequencePROTEIN FROM PLASMID INSERT
LEKRAS

SEQ ID NO: 49 - protein, 14 aa, Artificial Sequence
  Description of Artificial SequencePROTEIN FROM PLASMID INSERT
LEKRHHHHHHGSYV

(19) United States
(12) Patent Application Publication
(10) Pub. No.: US 2004/0132098 A1
(43) Pub. Date: Jul. 8, 2004

(54) Immunological binding

(75) Inventors: Steven Howell, Bedford (GB); Cornelis P.E. van der Logt, Bedford (GB); Stephen Wilson, Bedford (GB)

Correspondence Address: MORGAN LEWIS & BOCKIUS LLP, 1111 PENNSYLVANIA AVENUE NW, WASHINGTON, DC, 20004, US

(73) Assignee: UNILEVER PATENT HOLDINGS B.V.

(21) Appl. No.: 10/608,540

(22) Filed: Jun. 30, 2003

Related U.S. Application Data
(63) Continuation of application No. 09/477,562, filed on Jan. 4, 2000, now abandoned.

(30) Foreign Application Priority Data
Jan. 5, 1999 (EP) 99300058.7

(51) Int. Cl. G01N 33/53

(52) U.S. Cl. 435/7.1

(57) ABSTRACT

A process for producing an immunoadsorbant material, a surface of which comprises a molecule which incorporates at least a binding site of an antibody, which process comprises the step of exposing a solid surface of a material to a solution of a molecule which incorporates at least the binding site of an antibody such that the molecule adsorbs onto said surface, characterised in that said molecule is an antibody fragment attached through a covalent chemical bond to a second protein which is not the remainder of the corresponding antibody.

### TECHNICAL FIELD

The present invention is concerned generally with the preparation of surfaces which have been functionalised or sensitised with immunological materials.

### BACKGROUND ART

There is considerable interest in processes which employ in vitro immunological binding i.e. the recognition of the binding site of an antibody for its binding partner; generally a corresponding antigen or a material comprising an epitope of that antigen. The expression “in vitro” is used herein in its broad sense to denote the utilisation of scientific or industrial apparatus and thus distinguishes from processes occurring in nature in the metabolism of a living organism.

There are a number of procedures that require the specific functionality and hence the binding site of an antibody to be attached to a solid surface after which the resulting “sensitized” solid surface is used to bind binding partner from a solution to which the surface is exposed. Such a procedure may be a test for the presence of the binding partner or it may be an assay procedure in which the binding of binding partner to the surface is not merely detected but is also measured quantitatively, thereby providing a measurement of the amount of binding partner which was present in the test solution.

Recognition processes within this general category can also be used as purification processes (e.g using immunoaffinity columns) in which the binding partner is selectively removed from the solution to which the sensitized solid surface is exposed and the binding partner is subsequently detached from the antibody binding site into a further solution.

Attachment of the binding functionality of an antibody to a solid surface may be done by exposing the solid surface to a solution containing the antibodies, in the form of whole antibodies, and allowing those antibodies to be adsorbed onto the solid surface, through non-specific binding mechanisms, thereby “sensitizing” the surface concerned with the specific binding function of the antibody.

Commonly the solid surface is provided by a hydrophobic material, for example, polystyrene, and adsorption onto that surface is brought about by absorption of hydrophobic regions of the antibody onto the hydrophobic surface. This is a general mechanism by which protein can adsorb to hydrophobic surfaces. When adsorption onto the solid surface takes place, there is usually some partial unfolding and denaturation of the protein. In addition to this, it is possible that the portion of the protein which adsorbs onto the surface will be close to the antibody binding site, with the consequence that its adsorption interferes with the conformation of the molecule at the binding site. Thus when antibodies adsorb onto a hydrophobic surface, it is normal that a substantial amount of activity is lost so that something less than 5% of the original activity remains. However, this is still sufficient to provide a useful amount of recognition and binding of binding partner by the antibody binding site, and for the overall process to be useful. By contrast with whole antibodies, if antibody fragments are adsorbed onto a solid surface the amount of specific binding affinity which remains is extremely low (possibly as little as 0.1% for certain fragments).

Notwithstanding this it would be attractive to utilise antibody fragments adsorbed onto a solid surface for in vitro immunological binding processes. This is, inter alia, because an antibody fragment can be expressed by a genetically modified organism and hence produced more easily than whole antibodies.

Attachment of an antibody fragment fused to an albumin-binding domain to a derivatised solid surface by means of a specific binding interaction, as opposed to non-specific adsorption, is described by Konig et al, J. Immunological Methods, 218, 1-2, 73-78 (1998). Similarly, the possibility of a reversibly binding antibody also possessing a non-immunoglobulin region such as a fusion protein binding to a solid support is mentioned, but not exemplified, in WO 93/25909. A disadvantage of processes involving specific binding interactions in the preparation of immunoactive surfaces is that they generally require the solid surface to which the antibody is attached to be derivatised in some way.

EP 0434317 (Joseph Crosfield & Sons) discloses the use of improved affinity purification media which preferably employ Fv antibody fragments. These can optionally have a hydrophobic ‘tail’ which can consist of as few as two amino acid residues, and in one preferred form comprise the 11-mer myc amino acid sequence.

### DISCLOSURE OF THE INVENTION

Broadly, the present invention proposes that the specific binding site is provided by an antibody fragment but this fragment is chemically attached, through covalent bonding, to additional protein. This protein is not however merely the remainder of the antibody from which the fragment comes. Since the antibody fragment is also a protein, the additional protein to which it is attached will be conveniently referred to below as a second protein.

Thus, in a first aspect of the present invention there is disclosed a process for producing an immunoadsorbant material, a surface of which comprises a molecule which incorporates at least a binding site of an antibody, which process comprises the step of exposing a solid surface of a material to a solution of a molecule which incorporates at least the binding site of an antibody such that the molecule adsorbs onto said surface, characterised in that said molecule is an antibody fragment attached through a covalent chemical bond to a second protein which is not the remainder of the corresponding antibody.

This utilisation of an antibody fragment chemically joined to a second protein has a number of advantages.

The presence of the second protein increases the overall size of the entire molecule, reducing the denaturation as compared with the antibody fragment alone, and making it more likely that non-specific adsorption to the surface will take place through portions of the protein remote from the antibody binding site and thus increasing the amount of activity which is retained after absorption. Additionally, the second protein can be readily selected such as to have good properties of adsorption to the surface, as compared with a corresponding whole antibody.

For instance it can be more hydrophobic, or smaller than, the whole antibody, thereby allowing the production of a more stable surface with a greater density of packing than can be achieved using native antibodies.

Thus, it is possible to obtain the advantages of using only an antibody fragment and the advantages of using a large molecular size, without disadvantages arising from use of a whole antibody e.g. cross-reactivity problems arising from the constant region of the antibody, or low production levels of active protein.

Protein Coupling

The antibody fragment and the second protein to which it is attached can be expressed as a single fusion protein by a genetically modified organism, in which case the antibody fragment is of course coupled to the second protein through a peptide bond. Such fusions may be more readily expressible in an active form than whole (multiple-chain) antibodies.

Alternatively the antibody fragment can be attached by chemical conjugation to a second protein which is produced separately. In this case the chemical bond between the antibody fragment and the second protein will almost always be something other than a peptide linkage.

It will be appreciated the second protein may have more than one antibody fragment attached to it, which antibody fragments may have the same of different antigen binding specificity.

Solid Surface

A variety of materials may provide the solid surface onto which non-specific absorption of the molecule occurs. One possibility is polystyrene, a material commonly used for microtitre plates. Further possibilities are other plastics including polypropylene, polyvinylchloride, nylon, polyester (marketed as Melinex by ICI) cotton, metals such as gold, silver and platinum, carbon, glass, silica and other inorganics such as silicon nitride. The materials may be in any form appropriate for the immunoadsorbant purpose for which they are intended e.g. plates, flasks, columns, beads, dipsticks etc. For instance, one material which may be used to provide the solid surface is nitrocellulose, in the form of a nitrocellulose membrane. A further possible material is beads of a hydrophobic polymeric latex, which in some diagnostic tests are sensitized and transported by flow of a sample solution.

Antibody Fragments

The antibody fragment may be any of the various antibody fragments described in literature (see e.g. Antibody Engineering (2nd Edition) (Ed. Carl A. K. Borrebaeck), 1995, Oxford University Press, New York). Thus the possibilities include an Fab fragment which contains both binding sites of an antibody connected together, or an Fv fragment containing only a single antibody binding site. An Fv fragment may consist of the light and heavy chains associated together but not chemically attached or it may be a so called single chain Fv fragment (scFv) in which both the light and heavy chains are present as parts of a single fusion protein.

An even smaller fragment is only one chain of a binding site, e.g. non-isolated heavy chain. Particularly preferred are the fragments derived from Camelidae (see e.g. WO 94/25591) such as the HCV fragment referred to below (sometimes called the VHH fragment).

The Second Protein

The second protein is desirably chosen to display non-specific affinity for the solid surface (a sticky protein). It is also desirable that it has a substantial molecular weight such as at least 3000, better at least 5000 Daltons. In some embodiments it may be preferable to use proteins with molecular weights approaching those of intact antibodies (150 kDa or so) but there is no requirement to do so.

In the present invention the second protein functions as a molecular ‘shock absorber’. Thus there is absolutely no requirement that it be catalytically active per se e.g. as an enzyme which converts one or more chemical substrates to products. Indeed catalytically inactive proteins are preferred, thereby avoiding any risk that they will interfere with the target material to be bound on the immunoabsorbant surface.

Serum albumin is known to be suitable as a sticky protein, and may be used as the second protein in this invention. Other proteins which may be used as the second protein are other globular proteins like; ovalbumin, hydrophobins, lactoglobulin and haemoglobin (minus haem). However, non-globular proteins may also be used providing they confer the benefit of stabilising the binding site of the first protein upon adsorption.

EP 0479600 and EP 0481701 disclose combinations of antibody fragments and therapeutic agents or liposomes respectively, optionally joined by means of a peptide spacer. Other publications (e.g. EP 0451972) disclose combinations of antibody fragments with ‘active ingredients’. However, in these disclosures the agents are generally not adsorbed onto solid surfaces to form immunoadsorbant materials.

Other Aspects and Embodiments of the Invention

In a further aspect of the present invention there is disclosed an immunoadsorbant material obtained as described above.

Generally this will retain, at least, greater than 0.1% of the specific binding activity of the antibody fragments, by which is meant the binding capacity of the fragments after adsorption onto the immunoadsorbant surface, when compared with the equivalent amount of unadsorbed fragments (which represents 100%). More preferably it retains greater than 0.2, 0.3, 0.4, 0.5% of the activity. This can be assessed, for instance, by the methods used in the Examples below, or methods analagous to these.

As shown in the Examples below, the binding capacity of the immunoadsorbant materials of the present invention is demonstrably higher than (e.g at least double, preferably between 2 and 10 times higher, or possibly more) that which can be obtained with ordinary fragments when compared weight for weight of protein. In terms of retention of activity of actual binding sites, this represents a yet higher enhancement. In Examples below, a 10 fold improvement in activity has been demonstrated for when comparing HCV with an equivalent protein amount of GFP-HCV bi-head of the present invention.

Surfaces may carry more than one different antibody fragment modified in accordance with the present invention. Fragments may be immobilised as a plurality of discrete regions (e.g. microdots) on the surface, or spread evenly over it.

In a further aspect of this invention there is provided use of a second protein, attached through a covalent bond to an antibody fragment which includes at least the binding site of the antibody (which second protein is not the remainder of the corresponding whole antibody) to enhance retention of the antibody's specific binding affinity, upon adsorption to a solid surface. Preferably the second protein is used in process for production of an improved immunoadsorbant material as discussed above.

In a yet further aspect of the present invention there is disclosed use of the materials discussed above, in an in vitro immunological recognition process.

Thus one embodiment of this aspect provides an immunological recognition process carried out in vitro, in which a solid surface is exposed to a solution of a molecule which incorporates at least the binding site of an antibody, and the molecule absorbs onto said surface, after which said surface is exposed to a solution for binding of a target material from said solution onto said antibody binding site attached to said surface, characterised in that said molecule is an antibody fragment attached through a covalent chemical bond to a second protein which is not the remainder of the corresponding antibody.

The target material is recognised by the antibody, and is a binding partner therefor. As will be understood by those skilled in the art, generally the target material will comprise an epitope corresponding to that specifically bound by the antibody from which the fragment was obtained. The target material may correspond to the antigen used to raise the original antibody.

The procedure in which the invention is employed may, as mentioned above, be a test for the presence of an binding partner, an assay procedure or a purification procedure or indeed any other procedure in which it is useful to immobilise the binding function of an antibody to a solid surface.

In particular, the procedure may be an enzyme linked immuno-specific assay procedure (ELISA).

Thus the invention may be utilise in diagnostic test kits. It may also be utilised to bring about absorption onto a surface, which may be the surface of fabric or a particulate material, so that the surface will then be able to capture molecules from solution. An example of this is the preparation of immunoaffinity columns. Also included are immunoadsorbant materials (e.g. latex beads) which can be targeted to particular antigens (e.g. on hair or skin). All of these materials form further aspects of the present invention.

Embodiments of this invention will now be described by way of example only.

### FIGURES

FIG. 1 shows the assay response of both the scFv3299-HSA latex and the scFv3299HisphilII latex to various concentrations of hCG (see 2.3 below).

FIG. 2 shows the localisation of adsorbed latex to dot blotted keratin on PVDF membranes. In the Figure, (1) represents latex adsorbed with anti-keratin monoclonal antibody; (2) used anti-keratin llama HCV; (3) uses anti-keratin HCV-HSA-anti-keratin HCV fusion protein (see 3.3 below).

FIG. 3 shows the amino acid sequence of a HIS6-GFP-HCV21-myc fusion (see 4.1 below).

FIG. 4 shows a comparison of the hCG-alakaline phosphatase binding ability of various antibody fragments and fusions (see 4.5 below).

### EXAMPLE 1

This example used an antibody with specific binding affinity for the human chorionic gonadotrophin (hCG) protein. The binding site of the antibody was provided in three forms. One was a monoclonal antibody. Another was a single chain Fv fragment (scFv) containing the binding site of the same antibody together with a peptide of about 20 amino acids. This is designated “scFv3299” herein. It was previously disclosed in WO 96/27612 as “scFv.kc”. The third form, embodying the invention, was this scFv fragment chemically conjugated to bovine serum albumen (BSA).

The monoclonal antibody and the scFv fragment derived from it were both obtained by published procedures (see e.g. Garni et al (1987) Hybridoma 6: 637). 2 mg of the scFv fragment was conjugated to 10 mg bovine serum albumin (BSA) using a proprietary kit (Pearce and Warriner Ltd). The kit was used in accordance with the suppliers recommended method. The kit provides for initial activation of the BSA with 1-ethyl-3-[dimethylaminopropyl] carbodiimide (EDC) and the activated material then couples with the scFv through free amine groups so as to form a peptide bond between the two molecules. The product of this reaction, consisting of scFv as the first protein and BSA as the second protein was passed through a dextran column. This separated, by gel filtration chromatography, the protein conjugate from the reaction solution. The protein conjugate was eluted from this column into phosphate buffered saline(PBS).

Samples of the monoclonal antibody, the scFv fragment and the above chemical conjugate were each diluted in PBS to give concentrations of 100, 10, 1, 0.1 and 0.01 micrograms of protein per millilitre. (Equal concentrations of protein do not signify equal activities. The scFv fragment had the lowest molecular weight and the highest number of binding sites per unit weight of protein.) A control solution was PBS without any antibody protein at all.

100 μl from each dilution of each protein was used to sensitize a well of a polystyrene microtitre plate (Greiner Hi-bind). This was done in triplicate. The plate was then incubated overnight at 4° C. and washed five times with PBS.

A quantity of hCG protein was chemically conjugated to alkaline phosphatase enzyme by means of a glutaraldehyde coupling method. This was carried out as follows: hCG (1 ml of a 2 mg/ml solution in PBS) and Alkaline Phosphatase (1 ml of a 10 mg/ml solution in PBS) were stirred at room temperature (10 minutes) in a Reacti™ vial to allow the reactants to mix. 1 ml of the sample was removed and placed into a fresh Reacti™ vial and monomeric glutaraldehyde (37.5 ml of a 10% solution in distilled water) was added and stirred at room temperature for three hours. The reaction was then quenched and the product stabilised by adding 25 ml of conjugate storage buffer containing 5% ovalbumin and 0.1% sodium azide made up to 50 mM Tris.HCl, pH 7.5.

The resulting conjugate was dissolved in PBST at 10 micrograms protein per ml. 100 μl of this conjugate was added to each well of the microtitre plate which was then incubated for one hour at 37° C. to allow binding of hCG protein to the sensitised microtitre plate. Next the plate was washed five times with PBST.

In the third stage 100 μl of a para-nitrophenol phosphate (PNPP) substrate in buffer was added to each well of the plate. The plate was incubated at room temperature for approximately 10 minutes during which time the bound phosphatase enzyme converted the PNPP substrate to a coloured product. The optical density of each well of the plate was read using a plate reader with a wavelength filter set to 410 nm.

It will be appreciated that because each well of the plate received an identical quantity of the HCG-phosphatase conjugate and received an equal quantity of the PNPP substrate, the intensity of colour is dependent on the number of functioning antibody binding sites in each well. The measured optical densities are set out in the following table. Each value is an average from three wells of the plate.

| | Micrograms of protein per millilitre | | | | |
|---|---|---|---|---|---|
| | 0.01 | 0.1 | 1 | 10 | 100 |
| scFv fragment | 0 | 0 | 0 | <0.1 | <0.1 |
| monoclonal | 0 | <0.1 | 0.2 | 0.25 | |
| conjugate (invention) | 0 | <0.1 | 0.6 | 1.1 | 1.4 |

It can be seen that the conjugate in accordance with the present invention gave much greater binding of HCG than did the monoclonal antibody which in turn gave greater binding than did the scFv antibody fragment, even though the fragment had the greatest number of binding sites per unit weight.

### EXAMPLE 2

This example compared the same scFv3299 fragment and a fusion protein of that fragment and human serum albumin(HSA). Both were absorbed onto latex beads and their activity when bound onto the latex beads was compared. It should be noted that the molecular shock adsorbing protein (HSA in this case) can be appended to the C-terminus of the binding domain (as in HSA-scFv3299) or the N-terminus (scFv3299-HSA), as described in more detail below.

2.1 Construction of scFv3299-HSA Fusion Proteins

Two oligonucleotide primer pairs were used to amplify HSA from the genomic DNA of a Pichia pastoris clone containing the HSA gene (Pichia Expression Kit Instruction Manual, Invitrogen Corporation, San Diego, Calif.) using the PCR technique. DNA restriction sites were introduced by the primers, which enabled the construction of two different types of scFv 3299-HSA fusions.

Primer pair 1 consisted of primers SW2 1 and SW22, used for the synthesis of the construct HSA-scFv3299HisphilII. Primer pair 2 consisted of primers SW23 and PCR392, used for the synthesis of the construct scFv 3299His-HSA.

Two amplification cocktails were made thus:

1.5 μg of each primer of pair

1.5 μl of 20 mM dNTP mix

15 μl Stratagene 1 Ox Taq polymerase buffer mix

0.75 μl (3.75 U) Taq polymerase

1 μl (2.5 U) Pfu polymerase

dH₂O to a final volume of 150 μl

Pichia pastoris colonies containing the HSA gene, grown on an MD agar plate, were “stabbed” with a cocktail stick and were added to the PCR mixes. The mixes were split into three equal portions and were immediately thermocycled using the following protocol:

94° C.—2 minutes, then 33 cycles of:

94° C.—1 minute

52° C.—1 minute

72° C.—1 minute

72° C.—10 minutes (last cycle only)

The products of the amplification step were run on a 1% agarose gel. Bands of the correct molecular weight were excised from the gel and were purified using gel extraction columns (Qiagen) as per the manufacturers instructions.

scFv 3299His-HSA

The HSA PCR product using primer pair 1 was digested with BamHI/EcoRI. The Pichia pastoris expression vector pPIC9.scFv3299Hisphil gene was digested with BamHI/EcoRI, BamHI/EcoRI vector DNA fragment and BamHI/BamHI insert fragment were isolated. BamHI/EcoRI digested HSA PCR fragment DNA was ligated into the BamHI/EcoRI vector yielding pPIC9.HSA. The nucleotide sequence of the cloned HSA fragment was verified via automated taq dye terminator sequence analysis. A set of 10 oligonucleotide primers were made for this purpose (see Table 1). The previously isolated BamHI/BamHI scFv3299HisphilII fragment was inserted into BamHI/dephosphorylated pPIC9.HSA vector thus yielding pPICscFv3299His-HSA.

HSA-scFv3299HisphilII:

The SfiI/EcoRI scFv3299HisphilII insert from pUC19.scFv 3299HisphilII was inserted into SacI/EcoRI opened pPIC9 vector together with SfiI/SacI HSA PCR fragment (primer set 2) yielding pPIC9.HSA-scFv3299HisphilII.

| | |
|---|---|
| Code | Sequence |

| | |
|---|---|
| SW21 | CAC CTG GGCCAT GGC CGG CTG GGC CCC TAA GCC |
| | TAA GGC AGC TTG ACT TGC AG |
| SW22 | GGG CTT GAT TGG AGC TCG CTC ATT CC |
| SW23 | GCA GGA TCC GAT GCA CAC AAG AGT GAG GTT GC |
| PCR89 | TGA GGA GAC GGT GAC CGT GGT CCC TTG GCC CC |
| PCR392 | GCA AAT GGC ATT CTG ACA TCC |
| HSA 5′-1 | TCG GCT TAT TCC AGG GGT GTG |
| HSA 5′-2 | TCC CCC GAT TGG TGA GAC CAG |
| HSA 5′-3 | AAA TGA TGA GAT GCC TGC TGA CTT G |
| HSA 5′-4 | ATG CGC TGT TAG TTC GTT ACA CC |
| HSA 5′-5 | GAA ACA TAC GTT CCC AAA GAG |
| HSA 3′-1 | CAC ACC CCT GGA ATA AGC CGA G |
| HSA 3′-2 | GGG GAG GTT TGG GTT GTC ATC |
| HSA 3′-3 | TGG CAG CAT TCC GTG TGG AC |
| HSA 3′-4 | ACA TTT GCT GCC CAC TTT TCC TAG |
| HSA 3′-5 | AAA AGC AGC GAA ATC ATC CAT AAC |

DNA sequence of oligonucleotides used in Examples.

Pichia pastoris GS115 was transformed with PmeI digested pPIC9.scFv3299His-HSA or pPIC9.HSA-scFv3299HisphilII.

Briefly:

P. pastoris GS115 cells were grown overnight at 30° C. in 500 ml YPD medium (1% Yeast Extract, 2% Peptone, 1% Glucose) to OD₆₀₀=1.4. The cells were spun and the pellet was washed with sterile distilled water before resuspending in 100 ml KDTT buffer (50 mM Potassium Phosphate pH 7.5, 25 mM DTT). After 15 minutes incubation at 37° C. the cells were pelleted (3 min. 3000 rpm) and resuspended in 100 ml ice-cold STM buffer (92.4 g Glucose/l, 10 mM Tris.HCL pH 7.5, 1 mM MgCl₂). After 5 washes with this buffer the cell pellet was resuspended in a final volume of 0.5 ml STM buffer. Approximately 2-5 μg DNA in 2 μl H₂O (digested pPIC9 constructs: DNA purified via Phenol/Chloroform extractions and EtOH precipitation) was mixed with 70 μl of fresh competent P. pastoris cells (on ice). The cells were electroporated in a 0.2 cm cuvette at 1.5 kV, 400, 25 μF in a BioRad Gene-Pulser. Immediately after electroporation, 1 ml of YPD medium was added to the cells. After recovery for 1 h at 30° C., the cells were pelleted and resuspended in 200 μL 1 M Sorbitol and plated out onto MD plates (1.34% YNB, 4×10⁻⁵% Biotin, 1% Glucose, 0.15% Agar). Colonies formed by transformed cells (His⁺) were visible within 48 hours incubation at 30° C. Transformed P. pastoris cells GS115 were selected essentially as recommended by the Invitrogen Pichia pastoris expression manual: The plates containing the His⁺ transformants were used to screen for the Mut⁺ and Mut^{S} phenotype as follows: Using sterile toothpicks, colonies were patched on both an MM plate (1.34% YNB, 4×10⁻⁵% Biotin, 0.5% MeOH, 0.15% Agar) and an MD plate, in a regular pattern, making sure to patch the MM plate first. Approximately 100 transformants were picked for each construct. After incubating the plates at 30° C. for 2-3 days the plates were scored. Colonies that grow normally on the MD plates but show little or no growth on the MM plates were classified as Mut_{S} clones.

Transformed and selected P. pastoris clones were induced to express recombinant antibody using the protocol outlined below:

1. Using a single colony from the MD plate, inoculate 10 ml of BMGY (1% Yeast Extract, 2% Peptone, 100 mM potassium phosphate pH 6.0, 1.34% YNB, 4×10⁻⁵% Biotin, 1% Glycerol) in a 50 ml Falcon tube.

2. Grow at 30° C. in a shaking incubator (250 rpm) until the culture reaches an OD₆₀₀=2-8.

3. Spin the cultures at 2000 g for 5 minutes and resuspend the cells in 2 ml of BMMY medium (1% Yeast Extract, 2% Peptone, 100 mM potassium phosphate pH 6.0, 1.34% YNB, 4×10⁻⁵% Biotin, 0.5% Glycerol).

4. Return the cultures to the incubator.

5. Add 20 μL of MeOH to the cultures after 24 h to maintain induction. After 48 h harvest the supernatant by removing the cells by centrifugation.

Supernantants of scFv3299-HSA and HSA-scFv3299 expressing Pichia pastoris clones were evaluated via:

a) 12.5% SDS-PAGE coomassie stain (as described Molecular Cloning: a Laboratory Manual: 2nd edition, Sambrook et al, 1989, Cold Spring Harbor Laboratory Press).

b) ELISA: 100 μl of 5 μg/ml hCG was used to sensitize Greiner microtitre plate wells overnight at 4° C. Expression supernatants were serially diluted two fold in PBST and were added to each well (after first washing 3× with PBST). The plates were incubated for one hour at 37° C. and were washed as before. 100 μl 1/2000 rabbit anti scFv.3299 serum in PBST was added to each well and was incubated as before. The rabbit immunoglobulin was detected using a Goat anti-rabbit-alkaline phosphatase conjugate (PNPP substrate).

c) Biosensor analysis using a CM5 sensor chip coated with approximately 1200 resonance units of hCG. Supernatants were diluted 1/33 in HBS buffer and 15 μl of each was injected into a Biacore X instrument, running at 1 Hz data collection rate at a flow rate of 15 μl/min. 100 mM HCl was used to regenerate the sensor chip between sample additions.

Both scFv 3299His-HSA and HSA-scFv 3299HisphilII constructs were shown to contain antigen binding activity, and were shown to be of the correct size when measured by SDS-PAGE.

Recombinant Pichia pastoris produced scFv3299-HSA fusion protein was purified on a human chorionic gonadotrophin agarose affinity column as follows. The column was loaded with scFv3299-HSA containing BMMY media (20 ml) under gravity and then washed with 10 ml phosphate buffered saline. Bound material was eluted in 1 ml aliquots by the addition of 5×1 ml of 40 mM glycine.HCl, pH 2.5. The pH of the eluted fractions was adjusted by the addition of 100 ml 1 M Tris.HCl, pH 8.0. SDS-PAGE was used to identify the fraction(s) containing purified scFv3299-HSA fusion protein. Fractions 2 and 3, containing the expressed fusion protein (results not shown), were pooled to give 2 ml of scFv3299-HSA fusion protein at approximately 200 mg/ml.

2.2 Preparation of Latex Adsorbed with scFv3299-HSA Fusion Protein and Respectively scFv3299HisphilII

Two samples of Duke blue latex (50 μl of 10% solids) were reverse pipetted into round bottomed eppendorf tubes and 10 mM borate buffer, pH 8.5 containing 0.01% (v/v) merthiolate (B buffer) was added (950 μl). The latex solutions were mixed and then centrifuged (10 000 g for 10 mm at room temperature). The supernatants were removed and the pellets vortexed. To one of the latex pellets 1 ml of 100 μg/ml scFv3299-HSA fusion protein (made up in B buffer) was added and to the other latex pellet 1 ml of 100 μg/mI scFv3299HisphilII (also made up in B buffer) was added. The protein latex solutions were vortexed, sonicated for 10 s with a sonic probe and then incubated f or 1 h at room temperature with constant mixing. Following the 1 h incubation, 50 μl of 200 mg/ml bovine serum albumin was added to each protein latex solution and further incubated at room temperature with constant mixing. After 30 min the solutions were centrifuged (10 000 g for 10 min at room temperature) and the supernatants discarded. The latex pellets adsorbed with scFv3299-HSA fusion protein and scFv3299HisphilII were each resuspended in 1 ml of B buffer and stored at 4° C. until required.

2.3 Analyses of Adsorbed Latecies

Adsorbed latecies were tested by incubating 10 μl of each latex with 50 μl of various concentrations of hCG made up in phosphate buffered saline containing 0.1% sodium azide at room temperature. After 5 min samples were ran on nitrocellulose strips upon which a monoclonal antibody (3468) recognising hCG had been plotted in a line. Latex adsorbed with scFv3299HisphilII, or scFv3299-HSA fusion protein, bound hCG and this complex was captured at the monoclonal antibody line. The density of latex at the capture line was determined by reading the nitrocellulose strips on an autoreader.

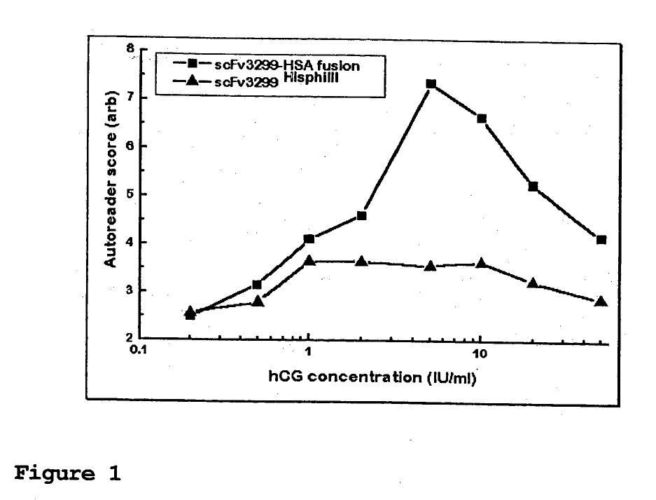

FIG. 1 shows the assay response of both the scFv3299-HSA latex and the scFv3299HisphilII latex to various concentrations of hCG. The latex adsorbed with the scFv3299-HSA fusion protein gave an increased assay response compared to that obtained with latex adsorbed with scFv3299HisphilII.

Since the two latices had been subjected to equivalent treatments, this shows that when the fusion protein adsorbed onto the latex solid surface, a greater amount of functioning, specific binding activity was conferred on the sensitized latex than when the scFv fragment was absorbed.

### EXAMPLE 3

### Activating Latex Surfaces by Adsorption of Antibody Fragment-Fusion Protein Specific for Keratin

3.1 Construction of Anti-Keratin Llama Antibody Fragment Constructs

Isolation of Gene Encoding the Anti Keratin HCV (HCV8)

Isolation of Gene Fragments Encoding Llama HC-V Domains From a llama immunized with keratin extract, a blood sample of about 200 ml was taken and an enriched lymphocyte population was obtained via Ficoll (Pharmacia) discontinuous gradient centrifugation. From these cells, total RNA was isolated by acid guanidium thiocyanate extraction (e.g. via the method described by Chomczynnski and Sacchi, 1987, Analytical Biochem 162:156-159). After first strand cDNA synthesis (e.g. with the Amersham first strand cDNA kit), DNA fragments encoding HC-V fragments and part of the long or short hinge region where amplified by PCR using specific primers:

| | PstI |
|---|---|
| V_{H} - 2B | 5′-AGGTSMARCTGCAGSAGTCWGG-3′ |
| S = C and G, M = A and C, R = A and G, W = A and T, | |
| | HindIII |
| Lam-07 | 5′-AACAGTTAAGCTTCCGCTTGCGGCCGCGGAGCTGGGGTCTTCGCTGTGGTGCG-3′ |
| | HindIII |
| Lam-08 | 5′-AACAGTTAAGCTTCCGCTTGCGGCCGCTGGTTGTGGTTTTGGTGTCTTGGGTT-3′ |

Upon digestion of the PCR fragments with PstI (coinciding with codon 4 and 5 of the HC-V domain, encoding the amino acids L-Q) and BstEII (located at the 3′-end of the HC-V gene fragments, coinciding with the amino acid sequence Q-V-T), the DNA fragments with a length between 300 and 400 bp (encoding the HC-V domain, but lacking the first three and the last three codons) were purified via gel electrophoresis and isolation from the agarose gel.

Construction of Saccharcmyces cerevisiae Expression Plasmids Encoding Llama HC-V Domains.

Plasmids pUR4547 and pUR4548 are Saccharomyces cerevisiae episomal expression plasmids, derived from pSY1 (Harmsen et al, 1993, Gene 125: 115-123). From this plasmid the PstI site, located in front of the GAL7 promoter was removed after partial digestion with PstI, incubation with Klenow fragment and subsequent blunt end ligation. After transformation the desired plasmid could be selected on the basis of restriction patern analysis. Subsequently, the BstEII site in the Leu2 selection marker was removed by replacing the about 410 bp AflII/PflMI fragment with a corresponding fragment in which the BstEII site was removed via a three step PCR mutagenesis, using the primers:

| | |
|---|---|
| PCR-A: | |
| | PflMI |
| BOLI 1 | 5′-GGGAATTCCAATAGGTGGTTAGCAATCG |
| | (BstEII) |
| BOLI 4 | 5′-GACCAACGTGGTCGCCTGGCAAAACG |
| PCR-B: | |
| | (BstEII) |
| BOLI 3 | 5′-CGTTTTGCCAGGCGACCACGTTGGTC |
| | AflII |
| BOLI 2 | 5′-CCCCAAGCTTACATGGTCTTAAGTTGGCGT |

PCR-A was performed with primers BOLI 1 and BOLI 4 and resulted in an about 130 bp fragment with the PflMI restriction site at the 3′-end and the inactivated BstEII site at the 5′-end. PCR-B was performed with primers BOLI 2 and BOLI 3 and resulted in an about 290 bp fragment with the AflII site at the 5′-end. The third PCR was with the fragments obtained from reaction A and B, together with the primers BOLI 1 and BOLI 2.

Finally, the about 1.8 kb SacI-HindIII fragment was replaced with synthetic fragments, resulting in the:

```
- SacI/HindIII fragment of pUR4547
      SacI
      GAGCTCATCACACAAACAAACAAAACAAAATGATGCTTTTGCAAGCCTTCCTTTTCCTTTTGGCTGGTTTTGCAGCCAAAATATCTGCGC
  1 ---------+---------+---------+---------+---------+---------+---------+---------+---------+ 90
      CTCGAGTAGTGTGTTTGTTTGTTTTGTTTTACTACGAAAACGTTCGGAAGGAAAAGGAAAACCGACCAAAACGTCGGTTTTATAGACGCG
          A H H T N K Q N K M M L L Q A F L F L L A G F A A K I S A Q
                                                                1-> SUC2 ss
                      PstI BstEII HindIII
      AGGTGCAGCTGCAGGAGTCATAATGAGGGACCCAGGTCACCGTCTCCTCATAATGACTTAAGCTT
91 ---------+---------+---------+---------+---------+---------+----- 155
      TCCACGTCGACGTCCTCAGTATTACTCCCTGGGTCCAGTGGCAGAGGAGTATTACTGAATTCGAA
          V Q L Q E S * * G T Q V T V S S * * L K L
              1-> HC-V cassette <-1
and
- SacI/HindIII fragment of pUR4548
      SacI
      GAGCTCATCACACAAACAAACAAAACAAAATGATGCTTTTGCAAGCCTTCCTTTTCCTTTTGGCTGGTTTTGCAGCCAAAATATCTGCGC
  1 ---------+---------+---------+---------+---------+---------+---------+---------+---------+ 90
      CTCGAGTAGTGTGTTTGTTTGTTTTGTTTTACTACGAAAACGTTCGGAAGGAAAAGGAAAACCGACCAAAACGTCGGTTTTATAGACGCG
          A H H T N K Q N K M M L L Q A F L F L L A G F A A K I S A Q
                                                              1-> SUC2 ss
                      PstI BstEII HindIII
      AGGTGCAGCTGCAGGAGTCATAATGAGGGACCCAGGTCACCGTCTCCTCAGAACAAAAACTCATCTCAGAAGAGGATCTGAATTAATGACTTAAGCTT
91 ---------+---------+---------+---------+---------+---------+---------+---------+---------+-------- 188
      TCCACGTCGACGTCCTCAGTATTACTCCCTGGGTCCAGTGGCAGAGGAGTCTTGTTTTTGAGTAGAGTCTTCTCCTAGACTTAATTACTGAATTCGAA
          V Q L Q E S * * G T Q V T V S S E Q K L I S E E D L N * * L K L
          1-> HC-V cassette <-1-> myc tail <-1
```

pUR4547 and pUR4548, respectively. Both plasmids contain the GAL7 promoter and PGK terminator sequences as well as the invertase (SUC2) signal sequence. In both plasmids the DNA sequence encoding the SUC2 signal sequence is followed by the first 5 codons (encoding Q-V-Q-L-Q) of the HC-V domain (including the PstI site), a stuffer sequence, the last six codons (encoding Q-V-T-V-S-S) of the HC-V domain. In pUR4547, this is followed by two stop codons, an AflII and HindIII site. In pUR4548, this sequence is followed by eleven codons encoding the myc-tag, two stop codons, an AflII and HindIII site.

Plasmids pUR4547 and pUR4548 are deposited at the Centraal Bureau voor Schimmelcultures, Baarn on 18th Aug. 1997 with deposition numbers: CBS 100012 and CBS 100013, respectively.

Upon digesting pUR4548 with PstI and BstEII, the about 6.4 kb vector fragment was isolated and ligated with the PstI-BstEII fragments of about 350 bp obtained as described above. After transformation of S. cerevisiae, via electroporation, transformants were selected from minimal medium agar plates (comprising 0.7% yeast nitrogen base, 2% glucose and 2% agar, supplemented with the essential amino acids and bases).

Screening for Antigen Specific HC-V Domains.

For the production of llama HC-V fragments with myc-tail, individual transformants were grown overnight in selective minimal medium (comprising 0.7% yeast nitrogen base, 2% glucose, supplemented with the essential amino acids and bases) and subsequently diluted ten times in YPGal medium (comprising 1% yeast extract, 2% bacto pepton and 5% galactose). After 24 and 48 hours of growth, the culture supernatant of the colonies was analyzed by ELISA for the presence of HC-V fragments which specifically bind to the antigens hCG, RR6, or keratin. In this case the presence of specifically bound HC-V fragments was detected by incubation with monoclonal anti-myc antibodies, followed by incubation with poly-clonal rabbit-anti-mouse conjugate with alkaline phosphatase. In this way a number of anti-hCG, anti-RR6 HC-V and anti-keratin fragments have been isolated, among which are:

anti-RR6:
R9 pUR4640
anti-hCG (alpha unit):
HI15 pUR4602
anti-keratin
HCV8

Construction of Episomal Expression Plasmids Encoding Anti-hCG/anti-RR6 Bispecific Bi-Heads.

In the anti-hCG HC-V fragment HI15 (anti-alpha-subunit), the PstI site was removed and a XhoI site was introduced via PCR, using the primers:

```
                                                      XhoI
                                                ** * **
  5′-GAATTAAGCGGCCGCCCAGGTGAAACTGCTCGAGTCWGGGGGA-3′
and
MPG159WB
                          BstEII
  3′-CCCTGGGTCCAGTGGCAGAGGAGTGGCAGAGGAGTCTTGTTT-5′
In this way the sequence:
                            PstI
CAG GTC CAG CTG CAG GAG TCT GGG
Q V Q L Q E S G
became
                                    XhoI
CAG GTG AAA CTG CTC GAG TCW GGG
Q V K L L E S G
```

Upon digesting the PCR fragments with XhoI and BstEII, the about 330 bp fragments were purified via agarose gel electrophoresis and isolation from the gel. The fragments were cloned into pUR4421 (see WO 94/25591) which was digested with the same enzymes, resulting in pJS3 (HI15). Subsequently, the about 420 bp EagI-HindIII fragments of pJS2 and pJS3 were isolated and ligated in the about 6.6 kb EagI-HindIII vector fragment of the pSY1 plasmid of which the PstI and BstEII sites were removes as described in Example 2.2. The resulting plasmid pJS8, was digested with BstEII and HindIII, after which the purified vector fragment was religated in the presence of a synthetic linker having the following sequence:

```
BstEII PstI HindIII
  <- MPG 160 WB (49) ->
GGTCACCGTCTCCTCACAGGTGCAGCTGCAGGAGTCACTGTAATGACTTAAGCTT
---------+---------+---------+---------+---------+----- 55
CCAGTGGCAGAGGAGTGTCCACGTCGACGTCCTCAGTGACATTACTGAATTCGAA
            <- MPG 161 WB (48) ->
  V T V S S Q V Q L Q E S L * * L K L
```

resulting in plasmid pJS10. Finally, this plasmid was digested with PstI and HindIII, after which the purified vector fragment of about 7.0 kb was ligated with the PstI-HindIII fragments of about 350 bp of pUR4640, encoding the anti-RR6 HC-V fragments R9, followed by the myc-tail. The resulting S. cerevisiae episomal expression plasmid pUR4621 encodes an anti-hCG—anti-RR6 bispecific bi-head preceded by the SUC2 signal sequence and followed by the myc-tail.

pUR4621: SUC2-HI15-R9-myc

Upon digesting these plasmids with XhoI and partially with BstEII, XhoI-BstEII fragments of about 0.7 kb can be isolated and subsequently cloned into the vector fragment of pUR4547 (digested with the same enzymes). In this way biheads can be obtained without the myc tail.

Obviously, expression vectors can be constructed in which different promoter systems, e.g. the constitutive GAPDH promoter or different signal sequences, e.g. the mating factor prepro sequence.

Construction of Integration Vectors for the Expression of Anti-hCG/anti-RR6 Bispecific Bi-Heads.

To allow the expression and secretion of the Llama bi-head constructs in P. pastoris, the gene encoding the bispecific construct was fused to the alpha-mating factor leader sequence in the commercially available P. pastoris expression vector pPIC9 (Invitrogen) The construction of the final expression vectors involved several cloning steps.

Step 1: The construction of the bispecific HCV expression vectors required the construction of two shuttle vectors, pPIC9N and pUC.HCVx2. For pUC.HCVx2 the HindIII/EcoRI polylinker of pUC19 was replaced with a synthetic HindIII/EcoRI fragment, destroying the original HindIII site, introducing a NheI site which allows the direct fusion to the alpha-Mating Factor leader sequence in pPIC9N, and introducing the XhoI and HindIII HCVx2 insertion sites.

Synthetic insert of pUC.HCVx2:

```
    ----A S Q V K L L E-----
AAGCTGCTAGCCAGGTGAAACTGCTCGAGCCCGGGAAGCTTGAATTC
            NheI XhoI HindIII
```

The synthetic linker was constructed by annealing the synthetic oligonucleotides PCR.650 and PCR.651.

PCR.650:
5′-AGCTGCTAGCCAGGTGAAACTGCTCGAGCCCGGGAAGCTTG-3′
PCR.651:
5′-AATTCAAGCTTCCCGGGCTCGAGCAGTTTCACCTGGCTAGC-3′

The XhoI/HindIII gene fragments encoding the bispecific HCV fragments were excised from pUR4621 (see Example 3.1) and inserted into the XhoI/HindIII opened pUC.HCVx2 shuttle vector, thus yielding the intermediate construct pUC.HCV21. For pPIC9N the XhoI/EcoRI polylinker of pPIC9 (Invitrogen) was replaced with a synthetic XhoI/EcoRI fragment which introduces a NheI restriction site immediately downstream of the alpha-Mating Factor leader sequence.

Synthetic insert of pPIC9N:

```
  L E K R A S-----
CTCGAGAAAAGAGCTAGCCCCGGGGAATTC
  XhoI NheI EcoRI
```

The new insert was constructed by annealing the synthetic oligonucleotides PCR.648 and PCR.649

| | |
|---|---|
| PCR.648: | 5′-TCCAGAAAAGAGCTAGCCCCGGGG-3′ |
| PCR.649: | 5′-AATTCCCCGGGGCTAGCTCTTTTC-3′ |

Step2: The final expression vectors were constructed via a three point ligation. The BamHI/NheI fragment from pPIC9N which contains the alpha-Mating Factor encoding sequence and the NheI/EcoRI HCVx2 insert from pUC.HCV21 were cloned together into a BamHI/EcoRI opened pPIC9 vector. This resulted in the isolation of the P. pastoris transformation and expression vector pPIC.HCV21 respectively.

HSA-HCV8HisphilII Intermediate.

The SfiI/EcoRI scFv3299HisphilII insert from pPIC9.HSA-scFv 3299HisphilII was removed and replaced by the SfiI/EcoRI HCV8-HisphilII fragment from pUC19.HCV8HisphilII. The resulting pPIC9.HSA-HCV8HisphilII construct was used in the construction of pPIC9.HCV8-HSA-HCV8HisphilII.

HCV8-HSA Intermediate.

The XhoI/BamHI HCV8-HisphilII encoding fragment from pUC19.HCV8-HisphilII was inserted together with the SacI/XhoI aMF leader sequence encoding fragment from pPIC9 into SacI/BamHI opened pPIC9.scFv3299-HSA (thus removing the aMF-scFv3299 fragment) thus yielding the pPIC9 HCV8-HSA construct.

HCV8-HSA-HCV8hisphilII.

pPIC9 HCV8-HSA-HCV8hisphil was constructed by inserting the SacI/XbaI aMF-HSA encoding fragment from pPIC9.HCV8-HSA in a 3 point ligation reaction together with the XbaI/EcoRI HSA-HCV8 encoding fragment from pPIC9 HCV8-HSA into SacI/EcoRI opened pPIC9.

Pichia pastoris was transformed with SacI opened pPIC9.HCV8-HSA-HCV8HisphilII essentially as described under 2.1. Individual colonies were evaluated for production of the recombinant protein using SDS-PAGE and Biosensor analysis: An NTA sensor chip was used to capture the His(6) tagged expression products from culture supernatants (prepared as described in the user manual). The samples were diluted 1/33 in HBS buffer and 15 μl of each was injected into a Biacore X instrument, running at 1 Hz data collection rate at a flow rate of 15 μl/min. 500 μM NiSO₄ in HBS buffer was used to prime the sensor surface prior to sample addition and 0.35M EDTA was used to regenerate the sensor chip after each sample.

Specific keratin binding activity was detected using a CM5 sensor chip loaded with approximately 1200 resonance units of keratin. Supernatants were diluted 1/33 in HBS buffer and 15 μl of each was injected into a Biacore X instrument, running at 1 Hz data collection rate at a flow rate of 15 μl/min. 100 mM HCl was used to regenerate the sensor chip between sample additions.

Purification of Pichia pastoris expression supernatants was via IMAC chromatography using commercially available NTA-agarose (Qiagen corporation). Culture supernatant was passed over 10 ml of resin packed into a 1.5 cm diameter column at 1 ml/min. The column was washed with 10 volumes of PBS and was eluted using 0.5 M imidazole in PBS. Eluted protein was dialyzed using 2×2.51 volumes of PBS.

3.2 Preparation of Latecies Adsorbed with an Anti-Keratin HCV8, a HCV8-HSA-HCV8 Fusion Protein and an Anti-Keratin Antibody

Three samples of Duke blue latex (50 ml of 10% solids) were reverse pipetted into round bottomed eppendorf tubes and 10 mM borate buffer, pH 8.5 containing 0.01% (v/v) merthiolate (B buffer) was added (950 ml). The latex solutions were mixed and then centrifuged (10 000 g for 10 min at room temperature). The supernatants were removed and the pellets vortexed. To one of the latex pellets 1 ml of 1 mg/ml anti-keratin HCV8 (made up in B buffer) was added. To a second latex pellet 1 ml of 1 mg/ml HCV8-HSA-HCV8 fusion protein (also made up in B buffer) was added. To the third latex pellet 1 ml of 1 mg/ml anti-keratin antibody (also made up in B buffer) was added. The protein latex solutions were vortexed, sonicated for 10 s with a sonic probe and then incubated for 1 h at room temperature with constant mixing. Following the 1 h incubation, 50 ml of 200 mg/ml bovine serum albumin was added to each protein latex solution and further incubated at room temperature with constant mixing. After 30 min the solutions were centrifuged (10 000 g for 10 min at room temperature) and the supernatants discarded. The latex pellets adsorbed with anti-keratin HCV8, HCV8-HSA-HCV8 fusion protein and anti-keratin antibody were each resuspended in 1 ml of B buffer and stored at 4° C. until required.

3.3 Analyses of Adsorbed Latecies

A solubilised keratin solution (0.5 ml) in 2% (w/v) SDS was dialysed against 500 ml of phosphate buffered saline containing 0.1% (w/v) sodium azide overnight at 4° C. The dialysate was then dot blotted (2 ml) onto PDVF membrane, after first wetting the membrane in methanol and then washing it in PBSA. Dot blots of keratin were incubated in a solution of latex (20 ml) made up in PBSA (1.98 ml) with gentle mixing for 2 h at room temperature. The membrane was washed by placing in 2 ml PBSA with gentle mixing for 5 mins and then air dried.

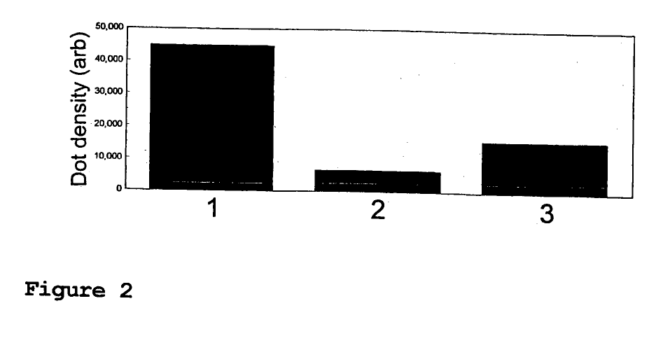

FIG. 2 shows that more latex adsorbed with anti-keratin HCV-HSA-anti-keratin HCV fusion protein (HCV8-HSA-HCV8) is localised to dot blotted keratin than latex adsorbed with anti-keratin HCV.

### EXAMPLE 4

### Activating Polystyrene Surfaces by Adsorption of Llama Antibody Fragment-Fusion Proteins Specific for hCG

4.1 Construction of a GFP-HCV Bihead Fusion Protein

The construction of the final pPIC-HIS6-GFP-HCV21-myc expression vector involved several cloning steps and the following plasmids as starting material:

pEGFP-N2 (CLONTECH, Genbank accession nr: U57608)

pPIC9 (Invitrogen)

pUC19 (New England Biolabs)

pPIC.HCV21. (See above).

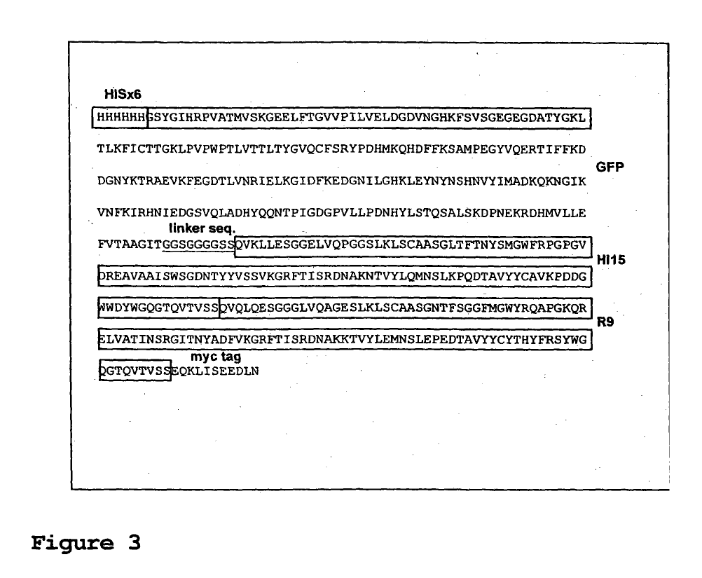

To allow the expression and secretion of the HIS6-GFP-HCV21-myc fusion constructs in P. pastoris, the gene encoding the HIS6-GFP-HCV21-myc construct (FIG. 3) was fused to the alpha-mating factor leader sequence in the commercially available P. pastoris expression vector pPIC9 (Invitrogen). The construction of the final expression vectors involved several cloning steps resulting in two intermediate vectors, pPIC9-HIS6 and pPIC-HIS6-GFP. For pPIC9-HIS6 the XhoI/SnaBI fragment of the leader peptide in pPIC9 was removed and replaced with a synthetic XhoI/SnaBI fragment, replacing the deleted leader sequence fragment, plus fusing it to a 6×HIS sequence.

Synthetic insert of pPIC9-HIS6:

---L E K R H H H H H H G S Y V---
CTCTCGAGAAAAGACATCACCATCACCATCACGGCTCTTACGTACAG
XhoI SnaBI

The synthetic linker was constructed by annealing the synthetic oligonucleotides PCR.529 and PCR.530

PCR.529:
5′-TCGAGAAAAGACATCACCATCACCATCACGGCTCTTAC-3′
PCR.530:
5′-GTAAGAGCCGTGATGGTGATGGTGATGTCTTTTC-3′

The expression vector pPIC9-HIS6-GFP was constructed by opening the pPIC9-HIS6 vector with SnaBI/NotI, thus removing the polylinker sequence from the vector, and replacing it with the SmaI/NotI GFP encoding gene sequence from pEGFP-N2.

The final expression vector pPIC9-HIS6-GFP-HCV21-myc was constructed in a 3 point ligation reaction linking the XhoI/XbaI HIS6-GFP PCR fragment from pPIC9-HIS6-GFP (using PCR.393 and PCR.689) to the NheI/NotI HCV21-myc fragment from pPIC9-HCV21-myc, and cloning it into XhoI/NotI opened pPIC9.

PCR.393:
5′-GACTGGTTCCAATTGACAAGC-3′
PCR.689:
5′-ATCGAATTCTCTAGATCCACCGCCTCCAGAACCGCCAGTGATCCC
GGCGGCGGTCACGAA-3′

4.2 Expression and Purification of GFP-HCV bi-head fusion Protein

BglII opened pPIC-HIS6-GFP-HCV21-myc was used to transform P. pastoris cells as described in section 2.1.

The crude supernatants were tested for the presence of HC-V bihead fragment via analysis on 12% acrylamide gels using the Bio-Rad mini-Protean II system.

Bispecific binding activity shown via ELISA as follows:

1) 96 well ELISA plates (Greiner HC plates) were activated overnight at 37 C with 200 μl/well of the BSA-RR6 conjugate (see example 1) in PBS.

2) Following one wash with PBST the wells were incubated for 1 hour at 37 C with 200 μL blocking buffer per well. Blocking buffer: 1% BSA in PBS-T

3) Serial dilutions of test samples (100 μL) were mixed with equal volumes of blocking buffer and added to the sensitised ELISA wells. Incubated at 37 C for 1-2 hours.

4) 200 μL hCG-AP conjugate in blocking buffer was added to each well in which the hCG was coupled to the alkaline phospathase via glutaraldehyde coupling.

5) Following one wash with PBST captured hCG-AP was detected by adding 100 μl/well pNPP substrate (1 mg/mL pNPP in 1 M diethanolamine/1 mM MgCl₂).

The crude supernantants were tested for the presence of GFP activity by diluting the supernatants (10 ml) were to 100 ml in PBSTA. These were then analysed on a Perkin Elmer fluorimeter with excitation at 488 nm and emission detected at 509 nm.

The culture supernatant (200 mL, pH 6-8) was clarified through a 0.45 m low protein binding cellulose acetate filter (Nalge Nunc Intl.), applied to a Ni-NTA Superflow column (5 mL, Qiagen Ltd, UK) at 2 mL/min, and washed with PBSA until the absorbance at 280 nm reached baseline. Elution with a linear gradient of 0-500 mM imidazole over 5 column volumes was followed by immediate buffer exchange by passage down a column of G-25 Sepadex (150 mL bed volume, Pharmacia) pre-equilibrated with PBSA, collecting 4 mL fractions. Peak fractions were assayed by SDS-PAGE and ELISA then combined and freeze dried in aliquots.

4.3 Construction of a HCV115-HSA-HCV115 (Bihead) Fusion Protein Vector

Construction of pPIC9.HCV115-HSA-HCV115HisphilII was essentially as described for HCV8-HSA-HCV8 in section 3.1. The intermediate vector pUC19.HCV115HisphilII had to be constructed in addition to those described in 3.1.

4.4 Expression and Purification of HCV115-HSA-HCV115 Fusion Protein

Expression and purification of recombinant HCV115-HSA-HCV115Hisphil was as described for the HCV8-HSA-HCV8 fusion protein. Evaluation of recombinant antibody was also as described, except that antigen recognition was assessed using a Biacore CM5 biosensor chip sensitized with hCG (as for the scFv3299-HSA constructs).

4.5 Analysis of Llama Antibody Fragment Fusion Proteins Adsorbed to a Polystyrene Surface

A Greiner Hi-Bind microtitre plate was sensitised overnight at room temperature using 100 μg/ml of the constructs in PBS in triplicate. The plate was then washed four times with PBS containing 0.1% Tween 20 (PBST) and each well was incubated with 100 μl of a hCG-alkaline phosphatase conjugate diluted 1 in 300 in PBST for 1 hour at room temperature. The plate was then washed as before and 200 μl Sigma 104 Phosphatase (Sigma Chemical Company) was diluted in substrate buffer as described by the manufacturer (Sigma). Following a 30 minute incubation at room temperature the optical density at 410 nm of each well was read on a plate reader.

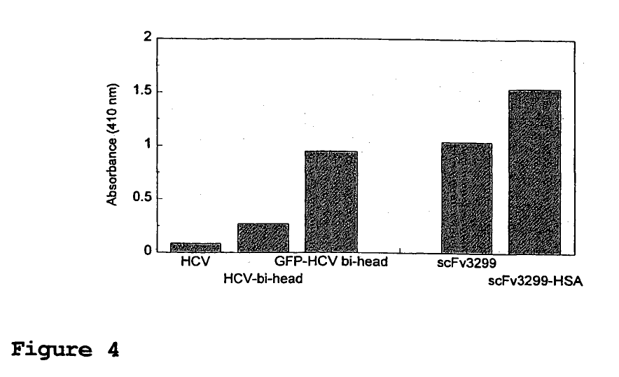

FIG. 4 shows that additions of molecular shock absorber mass to HCV results in an increased activity of alkaline phosphatase that is detected. As alkaline phosphatase is linked to captured hCG this reflects the binding activity of the adsorbed protein. Thus, addition of extra protein mass to HCV (and to scFv3299) provides a molecular shock absorber effect so increasing the activity of the adsorbed binding domain.

1. A process for producing an immunoadsorbant material, a surface of which comprises a molecule which incorporates at least a binding site of an antibody, which process comprises the step of exposing a solid surface of a material to a solution of a molecule which incorporates at least the binding site of an antibody, such that the molecule adsorbs onto said surface, characterised in that said molecule is an antibody fragment attached through a covalent chemical bond to a second protein which is not the remainder of the corresponding antibody.

2. A process as claimed in claim 1, wherein said molecule is a fusion protein whereby said antibody fragment is attached to said second protein through a peptide bond.

3. A process as claimed in claim 1, wherein said antibody fragment is chemically conjugated to said second protein.

4. A process as claimed in any one of the preceding claims wherein the material is selected from: polystyrene; polypropylene; polyvinylchloride; nylon; polyester; cotton; a metal; carbon; glass; silica; nitrocellulose; latex.

5. A process as claimed in any one of the preceding claims, wherein the second protein has a molecular weight of at least 3000 or 5000 Daltons.

6. A process as claimed in any one of the preceding claims wherein the second protein is catalytically inactive.

7. A process as claimed in claim 6, wherein the second protein is selected from serum albumin; ovalbumin; a hydrophobin; lactoglobulin; haemoglobin; GFP.

8. Use of a protein which is attached through a covalent bond to an antibody fragment including at least the binding site of the antibody, where the attached protein is not the remainder of the corresponding whole antibody, to enhance retention of the antibody fragment's specific binding affinity, upon adsorption to a solid surface.

9. Use as claimed in claim 8 in a process for producing an immunoadsorbant material.

10. An immunoadsorbant material obtainable by the process of any one of claims 1 to 7.

11. An immunoadsorbant material as claimed in claim 10 having a specific binding activity of greater than 0.1, 0.2, 0.3, 0.4, or 0.5%.

12. An immunoadsorbant material as claimed in claim 10 or claim 11 which constitutes all or part of: a microtitre plate; a flask; an immunoaffinity column; a polymeric beads; a dipstick.

13. Use of a material as claimed in any one of claims 10 to 12 in an in vitro immunological recognition process.

14. Use as claimed in claim 13, wherein the immunological recognition process comprises the steps of:
(a) exposing a solution of a molecule which is an antibody fragment which incorporates at least the binding site of an antibody attached through a covalent chemical bond to a second protein which is not the remainder of the corresponding antibody, to a solid surface, whereby the molecule adsorbs onto said surface,
(b) exposing said surface to a solution for binding of a target material from said solution onto said antibody binding site attached to said surface.

15. Use as claimed in claim 13 or claim 14, wherein the immunological recognition process is an enzyme linked immuno-specific assay procedure (ELISA).

16. Use of a material as claimed in any one of claims 10 to 12 in a purification procedure.

17. A diagnostic test kit comprising a material as claimed in any one claims 10 to 12.
